(19) United States
(12) Patent Application Publication
(10) Pub. No.: US 2026/0345966 A1
(43) Pub. Date: Nov. 5, 2026

(54) CATHETER DELIVERY DEVICE FOR DELIVERY OF A GENE THERAPY VECTOR TO A TARGET REGION OF THE HEART

(71) Applicants: Staffan Holmin, Bromma (SE); Magnus Settergren, Stockholm (SE); University of Eastern Finland, Kuopio (FI)

(72) Inventors: Seppo Ylä-Herttuala, Kuopio (FI); Jaakko Lampela, Kuopio (FI); Juho Pajula, Kuopio (FI); Staffan Holmin, Bromma (SE); Magnus Settergren, Stockholm (SE)

(73) Assignees: Staffan Holmin, Bromma (SE); Magnus Settergren, Stockholm (SE); University of Eastern Finland, Kuopio (FI)

(21) Appl. No.: 19/484,727

(22) PCT Filed: May 14, 2024
(86) PCT No.: PCT/EP2024/063190

§ 371 Date: Nov. 14, 2025

Related U.S. Application Data
(60) Provisional application No. 63/502,218, filed on May 15, 2023.

(51) Int. Cl. A61M 25/10; A61M 25/00

(52) U.S. Cl. CPC A61M 25/10; A61M 25/0023; A61M 25/0043; A61M 25/0097; A61M 2025/1052; A61M 2025/1093; A61M 2202/0007; A61M 2202/04; A61M 2210/125

(57) ABSTRACT

A catheter delivery device for delivery of a gene therapy vector to a target region of a heart, the catheter includes: a handle part; a multi-lumen cannula adapted to be inserted into the anterior cardiac vein, the cannula having a proximal end connected to the handle part, and a distal end including a plurality of delivery openings along the length of the cannula. The cannula further including: an occlusion balloon arranged between the proximal end and the distal end; a main lumen for accommodating a guidewire and for delivery of the vector; a control lumen, accommodating a pullwire arranged between the handle part and the distal end, the pullwire being adapted for steering the distal end. The control lumen is fluidly connected to the occlusion balloon.

### CROSS-REFERENCE TO RELATED APPLICATIONS

The present application is the national phase entry of International Patent Application No. PCT/EP2024/063190, filed May 14, 2024, which claims priority to U.S. Provisional Patent Application No. 63/502,218 filed May 15, 2023, the entire contents of both are hereby incorporated by reference.

### TECHNICAL FIELD

The present disclosure relates to a catheter delivery device for delivery of a gene therapy vector to a target region of a heart for treating heart failure and other cardiac diseases, and related methods.

### BACKGROUND

Myocardial diseases are a major global health concern and a leading cause of death worldwide. Despite significant advances in medical treatments, the prevalence and incidence of heart failure continue to rise, highlighting the need for new and innovative approaches to address this unmet medical need. Gene therapy has emerged as a promising approach for treating cardiac diseases, as it can provide long-term and site-specific gene expression, potentially leading to improved clinical outcomes.

However, a major challenge in cardiac gene therapy is the inefficient delivery of vectors to the myocardium. Among the current percutaneous cardiac gene delivery methods, endomyocardial injections and pressure-regulated coronary venous infusions are the most used. While these methods have shown some success in clinical trials, they have several limitations. For example, endomyocardial injections provide only spatially very targeted transgene expression, whereas pressure-regulated coronary infusion produces transgene expression only in the territory of the LAD. Intracoronary and intrapericardial delivery are global methods but are limited in efficacy. Recirculating vectors, however, can improve the efficacy of coronary infusions.

Adenovirus (Ad) is a frequently used viral vector and has been shown to be safe in clinical trials. However, Ad-mediated transgene expression lasts only approximately two weeks after the gene transfer. On the other hand, adeno-associated virus (AAV) produces long-term transgene expression, making it an attractive vector for gene therapy applications.

Despite the promise of gene therapy, there is a lack of an efficient method for the global transduction of viral vectors, particularly to the left ventricular (LV) myocardium. Other prior art devices have been described, but improvements are necessary to establish more reliable methods and delivery devices for more effective therapies (for example, see US 2002/177867 A, U.S. Pat. No. 6,017,323 A, and US 2022/241552 A1). Thus, the prior art has addressed some needs, however, they are still inefficient, particularly in relation to their distribution and transduction efficiency onto the targeted tissue.

Therefore, there is a strong need for new treatments for heart failure, and gene therapy provides a promising new approach. The use of Ad and AAV vectors for cardiac gene transfers has shown promising results, but the development of more efficient methods for their delivery is desirable for the success of gene therapy. Improving the efficiency and specificity of gene delivery to in particular the LV myocardium could potentially lead to significant improvements in clinical outcomes for patients with myocardial diseases.

In conclusion, the efficiency of cardiac gene delivery remains a major obstacle preventing its success, but improvements need to be pursued in establishing more reliable methods, more effective therapeutic genes, and better delivery devices. With the continued development of gene therapy approaches and delivery methods, there is great potential for improving outcomes for patients with myocardial diseases.

### SUMMARY

The present inventors have realized how a catheter delivery device can be arranged for efficient delivery of a gene therapy vector to a target region of a heart, thereby meeting the unmet needs, and overcoming the drawbacks of the prior art devices, and increasing the precision, safety and efficiency.

The present disclosure, therefore, relates in a first aspect to a catheter delivery device for delivery of a gene therapy vector to a target region of a heart, the catheter comprising:
a handle part;
a multi-lumen cannula adapted to be inserted into the coronary veins, the cannula having a proximal end connected to the handle part, and a distal end comprising a plurality of delivery openings along the length of the cannula, the cannula further comprising:
   an occlusion balloon arranged between the proximal end and the distal end;
   a main lumen, configured for accommodating a guidewire and for delivery of the vector;
   a control lumen that is fluidly connected to the occlusion balloon, for inflation and deflation of the balloon;
   a pullwire arranged between the handle part and the distal end, the pullwire being adapted for steering the distal end.

Examples of the present disclosed catheter delivery device can be seen in the appended figures. For example, FIG. 1 shows a schematic illustration of different components of an example of the catheter delivery device that includes a handle part which may be used for deflecting the catheter delivery device.

Additionally, the handle part of the catheter delivery device may comprise one or more hubs for efficient attachment of further devices. For example, said hub(s) may be provided in the form of luer hubs. In one example the catheter delivery device comprises two luer hubs, a first luer hub may be arranged for introduction of a guidewire. The first luer hub may, as such, be arranged to be connected to a main lumen of the catheter delivery device.

In embodiments, the device may further comprise a second luer hub that may be arranged to control the occlusion balloon. For example, a further device, such as a syringe, may be attached through a luer system to the second luer hub in order to accurately control the inflation of the occlusion balloon. The catheter delivery device typically comprises a pullwire attached at a distal end of the cannula, arranged for steering of the distal end and the pullwire typically runs in, and thereby shares, the same lumen as for the control of the balloon. The pullwire may be controlled for example by a pulling mechanism via the handle part. There is typically a sealing connection where the pullwire, that is connected to the handle pulling mechanism, enters the balloon inflation/deflation control lumen.

In specific examples, the control lumen is arranged to accommodate the pullwire and to be fluidly connected to the occlusion balloon. Thus, the control lumen may have multiple purposes, and thereby, the main lumen may have a larger size, thereby allowing for rapid infusions. In specific examples, the main lumen may be rotationally asymmetrical, for example by having a main wall of varying thickness. A thicker part of the main wall may be arranged to accommodate the control lumen, which may have multiple purposes, as mentioned above.

The cannula that is connected to the handling part typically has a size of 4-5 Fr (outer diameter 1.333 mm to 1.667 mm), and may further have a length of approximately 110 cm. In addition, the cannula may have two lumens, a main lumen and a control lumen. In embodiments, the cannula may comprise or be formed by an asymmetrical wall, wherein the control lumen may be arranged within a part of said wall, allowing for a maximal main lumen size and thereby decreased hydraulic resistance and faster injection times.

The control lumen is typically arranged to accommodate the pullwire and to allow for controlling the inflation of the occlusion balloon. Moreover, the occlusion balloon may have an annular configuration around the cannula. In embodiments, the occlusion balloon may be formed in an ultra-compliant material, and may have a size of 2.75-4.5 mm in diameter in its expanded state. Further, the distal end of the cannula, after the balloon section, comprises multiple openings that are intended for a fast and larger distribution of therapeutic agent.

In a further aspect, the present disclosure relates to use of the catheter delivery device as disclosed herein for gene therapy of a target region of a heart. Gene therapy of a target region of the heart poses on the devices used for delivery of the gene therapy vector. The configuration of the delivery device may have a significant impact on the outcome of the gene therapy. Therefore, the present disclosure includes all benefits of the catheter delivery device as disclosed elsewhere herein.

In yet a further aspect, the present disclosure relates to a method of gene therapy of a target region of a heart, the method comprising the steps of:
a. positioning a coronary balloon catheter into a coronary artery, such as the proximal part of the right or one of the major branches of left coronary artery;
b. positioning a catheter delivery device into a target vein that is regionally corresponding to said coronary artery, such as the anterior cardiac vein, the middle cardiac vein or the left marginal vein;
c. positioning one or more balloon catheter(s) to target vein's major venous anastomoses, for example as identified by retrograde venogram of the target vein;
d. occluding the coronary artery by the coronary balloon catheter;
e. reducing the amount of blood in the target region by administering, within 10 s of occluding, a saline solution through the coronary balloon catheter;
f. occluding the target vein and its' major anastomose(s) by a balloon of the catheter delivery device, and thereafter retroinjecting a viral vector through the catheter delivery device main lumen, during a first predetermined time;
g. releasing the coronary artery and the anastomotic vein(s) by deflating the balloon of the balloon catheter, and after a second predetermined time, releasing the target vein by deflating the balloon of the catheter delivery device.

### BRIEF DESCRIPTION OF THE DRAWINGS

FIG. 1 is a schematic illustration of one embodiment of the presently disclosed catheter delivery device.

FIG. 2 is a schematic illustration of a cross section of one embodiment of the presently disclosed catheter delivery device, at a position along its axial length distal to the occlusion balloon.

FIG. 3 is a schematic illustration of a cross section of one embodiment of the presently disclosed catheter delivery device, at a position of the occlusion balloon.

FIG. 4 is a schematic illustration of a cross section of a part of one embodiment of the presently disclosed catheter delivery device, in a plane parallel to its axial length.

FIG. 5 is a flow chart outlining a method of gene therapy of a target region of a heart according to an embodiment of the present disclosure.

### DETAILED DESCRIPTION

In a first aspect, the present disclosure relates to a catheter delivery device for delivery of a gene therapy vector to a target region of a heart.

In embodiments of the present disclosure, the catheter delivery device is arranged for delivery of a gene therapy vector to a target region of a heart. The target region of the heart may for example comprise or consist of the myocardium of a part of the heart. The target region may additionally or alternatively be a structural and/or functional part of the heart, including the left ventricle and/or the right ventricle. Further, said part may additionally or alternatively include the left and/or right atrium.

Typically, the catheter delivery device is arranged for delivery of a gene therapy vector to a target region of a heart by a retrograde injection(s), into a coronary vein(s) distal to the coronary sinus, such as the anterior cardiac vein, middle cardiac vein and left marginal vein.

In embodiments of the present disclosure, the catheter delivery device comprises a handle part, that is arranged such that a user of the catheter delivery device may hold and/or control said device. The handle part may for example comprise deflection means, such that movements of the handle part steer, control, operate and/or reposition other parts of the catheter delivery device, for example a distal end of the catheter delivery device, the occlusion balloon, and/or other parts of said device.

The handle part may comprise one or more ports, such as luer ports, for introduction or extraction of devices and/or substances into parts of the catheter delivery device, such as one or more of its lumens. The one or more ports may alternatively or additionally be arranged with locking means for attaching further devices, such as a luer lock system, wherein the further device may aid in the operation of the catheter delivery system.

For example, said further device may be a syringe arranged for injection of a substance into a lumen of the cannula of the catheter delivery device. For example, a fluid that is arranged to inflate the occlusion balloon. Further, the syringe may comprise the gene therapy vector, and in this way be arranged to deliver said vector to a target region of the heart.

In embodiments of the present disclosure, the catheter delivery devices comprise a handle part that is attached to the proximal end of the cannula. The handle part may be used for providing a gripping means or otherwise a means for manipulation of any part of the cannula. As such, the handle part may facilitate the insertion of the cannula into the patient's vein, by a surgeon or a user of the catheter delivery device.

In accordance with one embodiment of the present disclosure, the handle part may be arranged to facilitate the insertion of the cannula, or a part thereof such as the distal part, of the presently disclosed catheter delivery device, at a desired location.

For example, the catheter delivery device may be arranged for insertion to the femoral vein and, in embodiments, arranged such that a distal end of the cannula may be positioned into the anterior, middle or left marginal cardiac vein. As described, said insertion may further be facilitated by the use of the handle part of the catheter delivery device. The insertion of the device into a vein distal to the coronary sinus, may be facilitated in part by the use of a guiding catheter.

In one embodiment of the present disclosure, the handle part comprises a first and a second hub.

The first hub may be arranged for the introduction of the guidewire into the main lumen, and/or for the introduction of the vector into the main lumen. In embodiments, the second hub is arranged to be in fluidic communication with the control lumen, such that the second hub may be used for injection and/or withdrawal of a fluid into the control lumen. In this way, the inflation of the occlusion balloon may be controlled. The first hub and/or the second hub may be luer hubs.

Typically, the catheter delivery device comprises a control lumen that is fluidly connected to the occlusion balloon, thereby allowing for inflation and deflation of the balloon. For example through the first hub.

The catheter delivery device may further comprise a pullwire arranged between the handle part and the distal end, the pullwire being adapted for steering the distal end. The pullwire may be arranged in a separate lumen, such as a pullwire lumen, that is arranged between the handle part and the distal end. However, in other examples, the pullwire may be provided in the control lumen, such that the control lumen has multiple purposes. In this way, it may be possible to maximize the inner size of the main lumen, and thereby allow for rapid infusions.

In embodiments, the control lumen comprises a pullwire arranged for controlling a distal end of the cannula. Typically the pullwire is attached at a part of the distal end of the cannula. Typically, there is a sealing mechanism, where the pullwire enters the control lumen. The opposite part of the pullwire may be attached to the handle, such that the handle may be used to control, steer and/or operate the distal end of the cannula, thereby facilitating the insertion of the catheter delivery device into a vessel proximate or at the target region.

In embodiments of the present disclosure, the catheter delivery device comprises a multi-lumen cannula, comprising at least two lumens, such as a main lumen and a control lumen. Typically, the presently disclosed catheter delivery device is adapted for insertion into a cardiac vein, such as the anterior, middle or left marginal cardiac vein.

Typically, at least the distal end of the cannula is inserted into said vein. In embodiments, the cannula comprises a proximal end that is connected to the handle part. The distal end of the cannula is opposite of the proximal end, and comprises a plurality of openings, located distal to the inflatable balloon Typically, said openings comprise or consist of one or more delivery openings and at least one main opening arranged for accommodating a guidewire. In embodiments, the delivery openings are distributed along a length of the distal end of the cannula, such as homogeneously distributed along at least a part of the distal end.

The cannula may further comprise at least one main opening arranged to accommodate a guidewire. The main opening may be at the distal tip of the distal end, as such the main opening may be along the axial length of the cannula, while the delivery opening may be arranged perpendicular to the axial length of the cannula.

In one embodiment of the present disclosure, the catheter delivery device comprises an occlusion balloon. Typically, said occlusion balloon is arranged around at least a part of the cannula, in embodiments, the occlusion balloon is arranged between the proximal part and the distal part of the cannula. The occlusion balloon is typically located towards the delivery openings of the device. The occlusion balloon may for example be arranged such that it abuts the distal end.

In embodiments, the occlusion balloon is arranged for occluding a part of a blood vessel at or near the target region. Typically, the occlusion balloon is arranged to occlude a proximal part of a major vein of the target region. In one example, the occlusion balloon has a size of more than 2.75 mm and/or less than 4.5 mm in diameter in an expanded state.

In one embodiment of the present disclosure, the catheter delivery device comprises a multi-lumen cannula. The cannula may for example comprise a main lumen and a control lumen. In embodiments, the main lumen is configured for accommodating a guidewire. Alternatively or additionally, the main lumen may be arranged for delivery of fluid, for example a fluid comprising or consists of a gene therapy vector. In embodiments, the control lumen may be arranged for accommodating a pullwire and for allowing a fluidic connection between the handle part and the balloon catheter, such that inflation and/or deflation of the balloon may be controlled through the handle part, such as an opening and/or a hub of the handle part, that is connected to said control lumen.

In one example, the control lumen accommodates a pullwire arranged between the handle part and the distal end. In embodiments, the pullwire is adapted for steering, controlling and/or manipulating the distal end, for example to facilitate advancement of the catheter delivery device through the body. Typically, the control lumen is fluidly connected to the occlusion balloon, thereby allowing for controlling the inflation and/or deflation of the occlusion balloon. Typically, there is a sealing mechanism where the pullwire enters the control lumen.

The presently disclosed catheter delivery device provides for reproducible, and efficient cardiac vein retroinjection for gene therapy of the heart. In one example, the catheter delivery device relies on a handle part and a multi-lumen cannula adapted to be inserted into a cardiac vein.

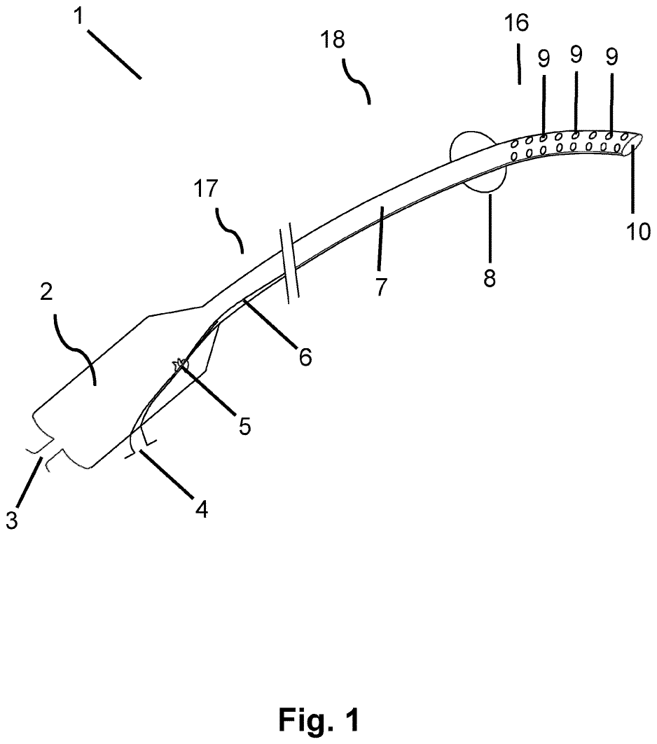

The dimensions of the catheter delivery device as seen in the schematic illustration of FIG. 1 is established primarily by the cannula size which has a dimension of possibly at least 4 Fr, less than 5 Fr in the French catheter scale (Fr) or possibly more than about 1.3 mm or less than about 1.7 mm as the outer diameter. Typically, at least for the distal end of the cannula. In embodiments, wherein said dimensions are for the entire cannula, and wherein the occlusion balloon is in the completely deflated state.

In embodiments, the occlusion ballon may have a size of more than 2.75 mm and less than 4.5 mm in diameter in its expanded state. The occlusion balloon may be a catheter delivery device. Further, the occlusion balloon may be formed from a compliant material, for example an ultra compliant material. Examples of material include polyurethane and/or silicon.

In embodiments of the present disclosure, the catheter delivery device comprises an elongated cannula which has a length of between 80 and 140 cm, between 100 and 120 cm, or around 110 cm. As mentioned above, in embodiments, the outer diameter is less than 2 mm, or than 1.5 mm. For example, the catheter delivery device may comprise a cannula having a size from 4 Fr to 5 Fr.

In addition, the cannula of the presently disclosed catheter delivery device may comprise at least two lumens, such as a control lumen and a main lumen, wherein the cannula has an asymmetrical wall along at least a part of its axial length, such as from the proximal end to the distal end of the cannula, in embodiments, the entire length of the cannula.

Typically, the control lumen is a smaller lumen, i.e. smaller lumen as compared to the main lumen. The control lumen may be arranged both for accommodating a pullwire and for allowing for control of the inflation and/or deflation of the occlusion balloon.

In one embodiment of the present disclosure, the handle part is attached to the proximal end of the cannula. In embodiments, the elongated cannula may comprise multiple lumens extending along the axial length of the cannula, such as within the cannula.

The multiple lumens of the cannula may be referred to as the main lumen (also called the main channel) and the control lumen (also referred to as the control channel). The cannula may be formed by a wall, wherein the outer part of said main wall defines the outer part of the cannula, and wherein the inner part of the main wall defines the main lumen.

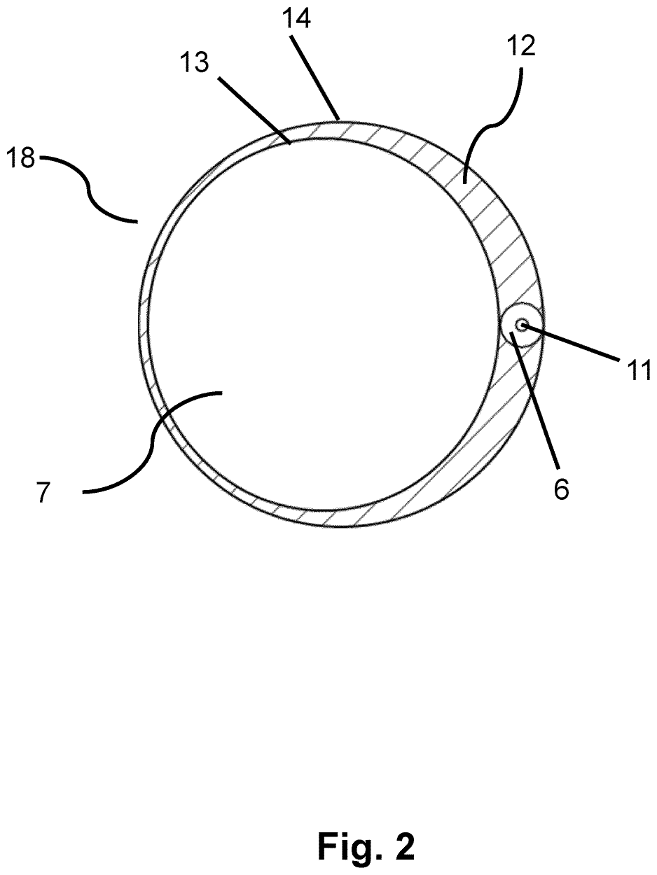

In particular, in one embodiment of the present disclosure, the cross-section of the main lumen and/or the cannula is rotationally asymmetric. The main wall of the cannula may for example have a non-uniform thickness, see for example FIG. 2. Such a rotationally asymmetric arrangement of the main lumen maximizes the size of the main lumen (in relation to the outer dimensions of the cannula) and thereby enables rapid infusions. In embodiments, said non-uniform wall may have a minimum distance between opposing parts of the wall of the main lumen, perpendicular to its length, to fit a guide wire. For example a guidewire with an outer diameter of 0.035 inches.

In embodiments, the cannula has a tubular shape, wherein the outer perimeter of the cross section is, at least in general, circular. For example, the occlusion balloon and openings of the cannula may act to make the cannula have a configuration that deviates from a perfect circular (at the outer side) tubular shape.

In one embodiment of the present disclosure, the outer shape of the cannula has a circular tubular shape. The lumens of the cannula may similarly have a circular cross section, or alternatively one or more, such as all, have a cross section that is non-circular. In embodiments of the presently disclosed catheter delivery device, the main lumen has a non-circular cross section. Said non-circular cross section may be a result of having the main wall of the cannula of non-uniform thickness. Typically, at least one part along the cross section of the main wall is thicker than the rest. Such an arrangement of the main lumen maximizes the size of the main lumen (in relation to the outer dimensions of the cannula) and thereby enables rapid infusions.

As such, the main lumen may have an asymmetric cross section or alternatively have for example be two-fold rotationally symmetric. In embodiments, the control channel is formed in the thicker part of the main wall of the cannula.

The outer shape of the cannula may for example have a circular cross section. At the same time, the main lumen may have a non-circular shape, for example an ellipsoidal shape. This may be a result of the main wall having a non-uniform thickness. Thus, the outer shape of the cannula may have a circular cross section. At the same time, the main lumen may have an ellipsoidal cross section, as a result of the varying thickness of the main wall of the cannula. The ellipsoidal cross section of the main lumen typically has two symmetry axes, e.g. a horizontal and a vertical symmetry axis as shown in FIG. 2. Notably, the horizontal symmetry axis overlaps with the centre of the circular cross section of the outer shape of the cannula, and/or the control lumen. At the same time, the vertical symmetry axis, of the ellipsoidal main lumen cross section, e.g. as shown in FIG. 2, does not overlap with the centre of the circular cross section of the outer shape of the cannula. Instead, the ellipsoidal may be displaced/biased towards the opposite side of the circular cross section of the outer shape of the cannula. In this way, a gradual increasing thickness of the main lumen wall can be generated. This simplifies the manufacturing process, and maximizes the internal volume of the main lumen, while simultaneously being provided in a format that makes the cannula suitable for delivering a payload, for example a gene therapy vector to a target region of a heart for treating heart failure and other cardiac diseases.

In one example, the cannula may have a circular outer cross-section, which encapsulates a main lumen with an ellipsoidal cross-section. This design choice is due to a variation in the thickness of the cannula's main wall. The ellipsoidal cross-section of the main lumen features two symmetry axes, a horizontal and a vertical axis. The horizontal symmetry axis aligns centrally with the circular outer cross-section, possibly overlapping with the control lumen's center.

The vertical symmetry axis of the main lumen's ellipsoidal cross-section may, in examples, not align with the center of the outer circular cross-section. Instead, it may be offset towards one side, resulting in a non-uniform wall thickness. This offset is strategic, enhancing the cannula's ability to accommodate internal structures or fluids by maximizing the available volume within the main lumen.

Further, the described asymmetry of the ellipsoidal main lumen within a circularly shaped cannula facilitates a simpler manufacturing process. The biased displacement of the ellipsoidal cross-section towards one side of the circular cross-section enables a gradual increase in wall thickness on the opposite side. This configuration optimizes material usage and simplifies the manufacturing steps involved.

Yet further, the unique cross-sectional arrangement of the cannula is specifically tailored to enhance its functionality in medical applications. The design is optimized for delivering specific payloads, such as gene therapy vectors, to targeted regions within the heart. This makes the cannula particularly suitable for treating conditions like heart failure and other cardiac diseases by ensuring precise delivery to the affected areas.

In one embodiment of the present disclosure, the main lumen may be used to accommodate a guidewire and arranged for delivery of a fluid. In embodiments of the present disclosure, the main lumen is arranged for accommodating a guidewire that is between 0.254 mm and 2.286 mm (0.01 to 0.09 inches) in diameter, between 0.356 mm and 0.965 mm (0.014 to 0.038 inches) in diameter, or about 0.889 mm (0.035 inches) in diameter.

The presently disclosed catheter delivery device may be arranged such that the inner size of the main lumen is maximized in relation to outer diameter of the cannula, such that the fluid may be delivered rapidly into or extracted from the target area and the fluid may include any type of buffer solution and/or vector, such as a vector for gene therapy. The maximized main lumen inner size may be a combined result of a rotationally asymmetric arrangement of the cross-section of the cannula, such as a non-circular cross section of the cannula, wherein for example the main wall of the cannula of non-uniform thickness, and wherein a thicker part of the main wall is arranged to accommodate a control lumen that combined the functionality of the pullwire and allows for inflation of the balloon.

A vector as used herein, is used to refer to, including but is not limited to, viral vectors, phages, plasmids, episomes, “naked DNA”, modified RNA or any of which desirably contains a transgene under the control of regulatory sequences that direct expression thereof in a target cell. In embodiments, the viral vector may include lentiviruses, picornavirus, adenoviruses, adeno-associated viruses, retroviruses, and baculoviruses among others.

In one embodiment of the present disclosure, the thickest part of the main wall of the cannula, along its cross-section, is at or near the control lumen. The control lumen may be used to accommodate a pullwire arranged between the handle part and the distal end of the cannula. In embodiments, the pullwire is attached to a part of the distal end of the guiding catheter. The pullwire is adapted for steering the distal end wherein the control lumen.

In addition, the control lumen comprises a balloon opening in fluidic connection with the occlusion balloon to allow the control of the inflation of the occlusion balloon. In one embodiment of the present disclosure, the occlusion balloon may be located between the proximal end and the distal end of the cannula, adjacent to the distal end, along the length of the cannula. In embodiments, the occlusion balloon may have a diameter of at least 2 mm in its expanded state, at least 2.75 mm, or between 2.75 mm and 4.5 mm. In one embodiment of the present disclosure, the occlusion balloon is made of a compliant material, such as polyurethane and/or silicone.

In one embodiment of the present disclosure, the distal end of the cannula comprises a main opening, located at a tip of the distal end. In embodiments, the distal end may comprise a plurality of delivery openings, distal to the occlusion balloon, extending perpendicular to the axial length of the cannula's distal end.

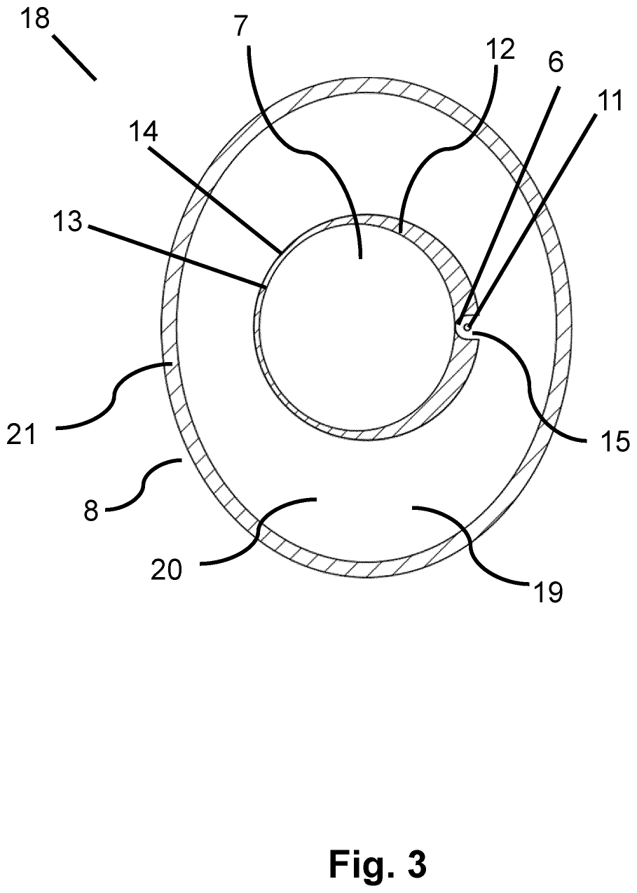

The delivery openings may be smaller in dimensions compared to the main opening and they may be distributed over the entire length distal to the occlusion balloon. In embodiments of the present disclosure, the delivery openings are uniformly distributed over the length of the distal end of the cannula as seen in FIG. 3. In embodiments of the present disclosure, the length of the distal end of the cannula is between 5 and 15 mm. The main opening and the multiple delivery opening may be used for the delivery of fluid, such as cardiac macromolecular complexes, pharmaceutical agents, and gene therapies to the heart, such as vectors.

In one embodiment of the present disclosure, the distal end of the cannula may be arranged to be positioned into any of the anastomotic veins of the target vein for gene therapy of a target region of a heart. In embodiments of the present disclosure, the catheter is arranged to infuse a payload at a flow rate of at least 0.01 mL/s, such at least 0.1 mL/s, such at least 1 mL/s, at an applied pressure of 10 kPa to the vector, i.e. an overpressure of 10 kPa applied to the vector.

In a further aspect, the present disclosure relates to use of the catheter delivery device as disclosed herein for gene therapy of a target region of a heart. Gene therapy of a target region of the heart poses on the devices used for delivery of the gene therapy vector. The configuration of the delivery device may have a significant impact on the outcome of the gene therapy. Therefore, embodiments of the present disclosure includes all benefits of the catheter delivery device as disclosed elsewhere herein.

In yet a further aspect, the present disclosure relates to a method of gene therapy of a target region of a heart, the method comprising the steps of:
a. positioning a coronary balloon catheter into a coronary artery, such as the proximal part of the right or one of the major branches of left coronary artery,
b. positioning a catheter delivery device into a target vein that is regionally corresponding to said coronary artery, such as the anterior cardiac vein, the middle cardiac vein or the left marginal vein;
c. positioning one or more balloon catheter(s) to target vein's major venous anastomoses, identified by retrograde venogram of the target vein; occluding the coronary artery by the coronary balloon catheter,
d. reducing the amount of blood in the target region by administering, within 10 second of occluding the coronary artery, a saline solution through the coronary balloon catheter;
e. simultaneous or subsequent to the preceding step, occluding the target vein by the catheter delivery device, and thereafter retroinjecting therapeutic vectors through the balloon during a first predetermined time,
f. releasing the coronary artery by deflating the balloon of the balloon catheter,
g. simultaneous to the preceding step, releasing the anastomotic vein(s) of the target vein,
h. after a second predetermined time, releasing the target vein by deflating the catheter delivery device.

In embodiments, introducer sheaths are used for introducing a coronary balloon catheter and a catheter delivery device. In embodiments, the introducer sheaths are connected to a suitable vein and a suitable artery for introduction of the coronary balloon catheter and the catheter delivery device respectively.

One introducer sheath may for example be placed in the femoral or radial artery for introduction of the coronary balloon catheter. A further introducer sheath may for example be placed in the femoral veins for introduction of the catheter delivery device.

The catheter delivery device is introduced into the anastomotic vein by a guiding catheter. In embodiments, the guidewire is used to guide the coronary balloon catheter into the proximal coronary artery.

In one embodiment of the present disclosure, the main anastomotic vein to the target vein is occluded simultaneously or just before the occlusion of the target vein. As the retroinjection of the therapeutic vector is performed the main anastomotic vein is released immediately right after.

In one embodiment of the present disclosure, the steps of the method of gene therapy may be repeated at least once, and where during said repetitions, the target vein may be a different vein located in the same target region.

In one embodiment of the present disclosure, the method comprises a step of reducing the amount of blood in the target region, just after the step of occluding the coronary artery. The step of reducing the amount of blood in the target region may comprise of administering a solution through the coronary balloon catheter and/or the catheter delivery device. In embodiments, the solution is at least administered to the arterial side of the heart, i.e. through the coronary balloon catheter. In embodiments, an isotonic saline solution is administered.

The present disclosure will in the following be described in greater detail with reference to the accompanying drawings. The drawings are exemplary and are intended to illustrate some of the features of the presently disclosed catheter delivery device and related methods, and are not to be construed as limiting to the present disclosure.

FIG. 1 shows a catheter delivery device (1) according to an embodiment of the present disclosure.

The catheter delivery device (1) is arranged for delivery of a gene therapy vector to a target region of a heart. The target region of the heart may for example comprise or consist of the myocardium of a part of the heart. The target region may additionally or alternatively be a structural and/or functional part of the heart, including the left ventricle and/or the right ventricle. Further, said part may additionally or alternatively include the left and/or right atrium

The catheter delivery device (1) comprises a handle part (2), that is arranged such that a user of the catheter delivery device may hold and/or control said device. The handle part comprises deflection means, such that movements of the handle part steer, control, operate and/or reposition other parts of the catheter delivery device, for example a cannula (18), including parts of the cannula such as its distal end (16) and/or its proximal end (17).

The handle part (2) comprises two hubs. A first hub (3) is located at the most proximal end of the handle part. A second hub (4) is located on one of the sides of the proximal handle part. The first hub and/or the second hub may be luer hubs.

The first hub (3) is arranged for the introduction of a guidewire into the main lumen (7), and/or for the introduction of a vector into the main lumen (7), such as a solution comprising the vector. The second hub (4) is arranged to be in fluidic communication with the control lumen (6), such that the second hub (4) can be used for injection and/or withdrawal of a fluid into the control lumen (6). In this way, the inflation of an occlusion balloon may be controlled.

The handle (2) further comprises an attachment mechanism (5) for controlling the distal end (16) of the cannula (18). In the example shown, the control lumen (6) is arranged for accommodating a pullwire and for controlling the inflation of an occlusion balloon (8) arranged around the elongated cannula. However, as disclosed elsewhere herein, the pullwire may be provided in a separate pullwire lumen.

Further, also as disclosed elsewhere herein, by accommodating the pullwire arrangement within the control lumen, that is also arranged for inflation and/or deflation of the balloon, the interior space of the cannula occupied by these features are reduced. Thereby, allowing for a maximized main lumen inner size, and as a result rapid infusions.

This effect may further be a combined result of having a rotational asymmetric arrangement of the cross-section of the cannula, wherein the main wall of the cannula has a non-uniform thickness, and wherein a thicker part of the main wall is arranged to accommodate a control lumen that is arranged to both accommodate the pullwire and allow for inflation of the balloon. Thus, the inner size of the main lumen is maximized in relation to the outer size of the cannula, thereby allowing for rapid infusions.

The distal end (16) of the cannula (18) comprises multiple delivery openings (9). The distal end of the cannula further comprises a main opening (10)

The delivery openings (9) are smaller in dimensions compared to the main opening (10) and they are further distributed uniformly over the length of the entire distal end (16). The length of the distal end of the cannula is typically between 5 and 15 mm and the length of the cannula is around 110 cm, from the proximal end (17) of the cannula to the distal end of the cannula (16). The main opening (10) and the multiple delivery openings (9), only a few is referenced in the figure) are arranged such that they can be used for delivery of a fluid comprising for example intracardiac macromolecular complexes, pharmaceutical agents, and/or gene therapeutic agent, to the heart, such as gene therapy vectors.

The first hub (3) is arranged to be in fluidic connection with the main lumen (7) of the cannula, which extends to the opening (10) at the distal tip of the cannula, at the distal end of the cannula (16). The main lumen is arranged to accommodate a guidewire and additionally arranged for the delivery of a fluid, as stated above. The main lumen (7) is arranged for accommodating a guidewire that is 0.035 inches in diameter.

FIG. 2 shows a cross-section of one embodiment of the catheter delivery device (1) of the present disclosure, at different positions along the axial length of the cannula (18). The cross-section is perpendicular to the device's axial length at a proximal end of the cannula (18). The main lumen (7) provides for a large channel for delivery of a fluid to the target site, and can accommodate a guidewire. The smaller control lumen (6) is arranged to accommodate a pullwire (11) and to allow for controlling the inflation of the occlusion balloon.

The cannula is formed by a main wall (12), wherein the outer surface (14) of said main wall defines the outer surface of the cannula, and wherein the inner surface (13) of the main wall defines the main lumen.

As can be seen, the cross-section of the main lumen (7) is rotational asymmetric, as the main wall (12) of the cannula has a non-uniform thickness. The non-uniform main wall (12) has a minimum distance between opposing parts of the wall of the main lumen, perpendicular to its length, that is sufficiently large to fit a guide wire that is 0.035 inches in diameter.

It can be seen that cannula has a circular tubular shape, as defined by the outer surface (14) of the main wall (12). The main lumen (7) however, has a non-circular cross section. Said non-circular cross section is a result of having the main wall (12) of the cannula of non-uniform thickness. It can further be seen that the control channel (6) is formed in a thicker part of the main wall (12) of the cannula (18).

FIG. 3 shows a cross-section of the cannula (18) of the catheter delivery device (1) in a plane perpendicular to its axial length at a position of the occlusion balloon (8). The main channel (7) is seen as defined by the inner surface (13) of the main wall (12). The outer surface (14) of the main wall has a circular cross section. The main wall has a non-uniform thickness, wherein a thicker part of the main wall (12) accommodates the control lumen (6).

The control lumen (6) comprises a balloon opening (15) that is in fluidic connection with a balloon cavity (20) of the occlusion balloon (8), as defined by the balloon wall (21). The balloon opening (15) and the control lumen (6) is arranged such that the inflation of the occlusion balloon (8) may be controlled from the handle part, such as from a device connected to a hub of the handle part.

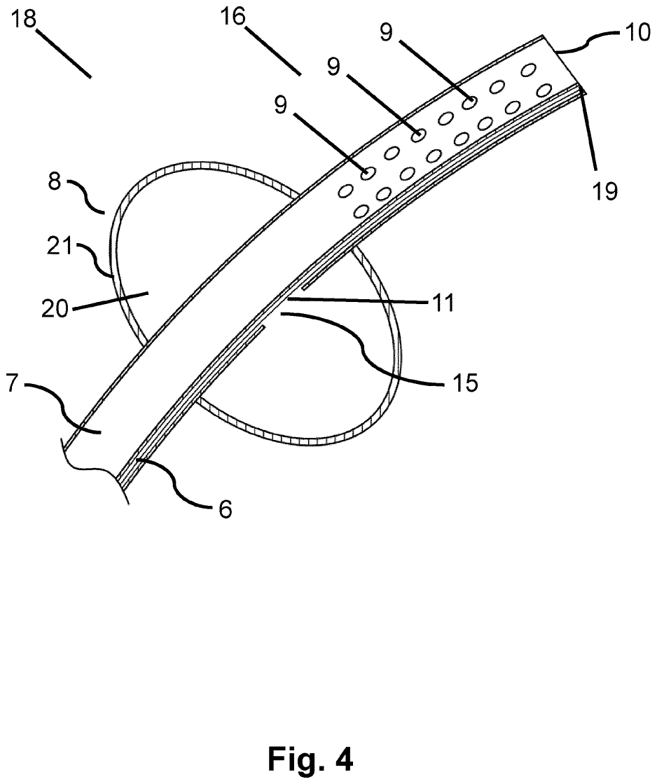

FIG. 4 shows a cross section of a part of the catheter delivery device, in a plane parallel to the axial length of the cannula (18). In particular, the cannula (18) is shown comprising a main lumen (7) and a control lumen (6) housing a pullwire (11). The control lumen has a balloon opening (15) such that the control lumen is in fluidic connection with the balloon cavity (20) of the occlusion balloon (8), as defined by the balloon wall (21).

The pullwire is attached by an attachment mechanism (19) at the distal end (16) of the cannula (18). The pullwire is arranged to steer, manipulate and control the cannula, such as the distal end, from the handle part. Facilitating the positioning of the catheter delivery device.

The distal end comprises a plurality of delivery openings. The delivery openings (9) are smaller in dimensions compared to the main opening (10) and they are further distributed uniformly over the length of the entire distal end (16). The length of the distal end of the cannula is typically between 5 and 15 mm. and the length of the cannula is around 110 cm, from the proximal end (17) of the cannula to the distal end of the cannula (16). The main opening (10) and the multiple delivery openings (9), only a few is referenced in the figure) are arranged such that they can be used for delivery of a fluid comprising for example intracardiac macromolecular complexes, pharmaceutical agents, and/or gene therapeutic agent, to the heart, such as gene therapy vectors.

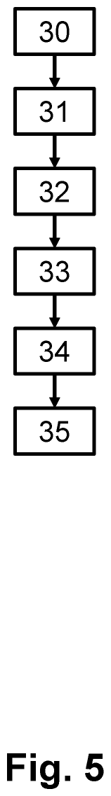

FIG. 5 shows a flowchart outlining an example of a method of gene therapy of a target region of a heart according to an embodiment of the present disclosure.

The disclosed method comprises a step of positioning a coronary balloon catheter into a coronary artery (30), such as the proximal part of the right or left coronary artery, wherein the coronary balloon catheter is arranged for occluding said coronary artery.

The disclosed method further comprises a step of positioning a catheter delivery device into a target vein (31). The target vein is selected to be regionally corresponding to the coronary artery, such as the anterior cardiac vein.

The disclosed method further comprises a step of occluding the coronary artery by the coronary balloon catheter (32).

The disclosed method further comprises a step of occluding the target vein by the catheter delivery device (33). This step may include retroinjecting a viral vector through the balloon during a first predetermined time.

The disclosed method further comprises a step of releasing the coronary artery by deflating the balloon of the balloon catheter (34).

The disclosed method further comprises a step of, after a second predetermined time, releasing the target vein by deflating the catheter delivery device (35).

The disclosed method may further comprise a step of reducing the amount of blood in the target region (not shown), just after the step of occluding the coronary artery. The step of reducing the amount of blood in the target region comprises administering a solution through the coronary balloon catheter and/or the catheter delivery device. The solution is administered to the arterial side of the heart, i.e. through the coronary balloon catheter.

One introducer sheath may for example be placed in the femoral artery for introduction of the coronary balloon catheter. A further introducer sheath(s) may for example be placed in the femoral vein(s) for introduction of the catheter delivery device. In specific examples, at least two sheaths to veins and one to artery may be used.

The catheter delivery device is introduced into the anastomotic vein by a guiding catheter. In embodiments, the guidewire is used to guide the coronary balloon catheter into the proximal coronary artery.

In one embodiment of the present disclosure, the main anastomotic vein to the target vein is occluded simultaneously or just before the occlusion of the target vein. As the retroinjection is performed the main anastomotic vein is released immediately right after.

In one embodiment of the present disclosure, the steps of the method of gene therapy may be repeated at least once, and where during said repetitions, the target vein may be a different vein located in the same target region.

### Figure Reference List

1 catheter delivery device
2 handle part
3 first hub
4 second hub
5 attachment mechanism
6 control lumen
7 main lumen
8 occlusion balloon
9 delivery openings
10 main opening
11 pullwire
12 main wall
13 inner surface of main wall
14 exterior surface of main wall
15 balloon opening
16 distal end of cannula
17 proximal end of cannula
18 cannula
19 attachment mechanism (pullwire)
20 balloon cavity
21 balloon wall
30 positioning a coronary balloon catheter into a coronary artery
31 positioning a catheter delivery device into a target vein
32 occluding the coronary artery
33 occluding the target vein
34 releasing the coronary artery
35 releasing the target vein

1-36. (canceled)

37. A catheter delivery device comprising:
a handle;
an elongated multi-lumen cannula extending from the handle and defining an outer circular cross-section;
a main lumen within the cannula, the main lumen having a non-circular cross-section and being positioned off-center relative to a central axis of the outer circular cross-section;
a main wall surrounding the outer circular cross-section, the main wall having a non-uniform thickness that results from the off-center non-circular shape of the main lumen;
a control lumen disposed within a thickened region of the main wall;
an occlusion balloon in fluid communication with the control lumen, the occlusion balloon being located at a distal portion of the cannula; and
a pullwire extending from the handle through the control lumen to the distal portion of the cannula and configured to deflect the distal portion of the cannula;
wherein the distal portion of the cannula includes a plurality of delivery openings for delivery of a therapeutic fluid.

38. The catheter delivery device of claim 37, wherein the handle comprises a first luer hub configured to provide access to the main lumen for introduction of a guidewire or the therapeutic fluid.

39. The catheter delivery device of claim 37, wherein the handle comprises a second luer hub in fluid communication with the control lumen, the control lumen being in fluid communication with the occlusion balloon through an opening in the control lumen.

40. The catheter delivery device of claim 37, wherein the elongated cannula has a length of between 80 and 140 cm.

41. The catheter delivery device of claim 37, wherein the occlusion balloon is located adjacent to the distal end, along the length of the cannula.

42. The catheter delivery device of claim 37, wherein the elongated cannula comprises a guidewire outlet at the distal portion of the cannula.

43. The catheter delivery device according to claim 37, wherein the occlusion balloon has, in an expanded state, a diameter between 2 mm and 4.5 mm.

44. The catheter delivery device of claim 37, wherein the pullwire is attached to a distal portion of the cannula.

45. The catheter delivery device of claim 37, wherein the occlusion balloon is made of a compliant polymeric material.

46. The catheter delivery device of claim 37, wherein the distal portion having the delivery openings has a length between 5 mm and 15 mm.

47. The catheter delivery device of claim 37, wherein the delivery openings are uniformly spaced along the distal portion of the cannula.

48. The catheter delivery device of claim 37, wherein the cannula has an outer diameter less than 2 mm.

49. The catheter delivery device of claim 37, wherein the non-circular main lumen has an ellipsoidal cross-section.

50. The catheter delivery device of claim 37, wherein the minimum distance between opposing parts of the main lumen wall perpendicular to its length is at least 0.035 inches.

51. The catheter delivery device of claim 37, wherein a thickest region of the main wall is located at or adjacent to the control lumen.

52. A method of delivering a therapeutic fluid to cardiac tissue, the method comprising advancing the catheter delivery device of claim 37 into a cardiac vein and delivering the therapeutic fluid through the distal delivery openings of the device.

53. The method of claim 52, further comprising inflating the occlusion balloon of the catheter delivery device during delivery of the therapeutic fluid.

54. The method of claim 52, further comprising deflecting a distal portion of the catheter delivery device using the pullwire.

55. The method of claim 52, further comprising positioning the distal portion of the catheter delivery device within the cardiac vein prior to delivering the therapeutic fluid.

56. The method of claim 52, wherein the therapeutic fluid is delivered by retrograde perfusion.
